(19) United States
(12) Patent Application Publication
(10) Pub. No.: US 2004/0011361 A1
(43) Pub. Date: Jan. 22, 2004

(54) Pressure monitoring means in divers' breathing apparatus

(76) Inventor: Alan Clarke, Lancashire (GB)

Correspondence Address: Samuels, Gauthier & Stevens LLP, Suite 3300, 225 Franklin Street, Boston, MA, 02110, US

(21) Appl. No.: 10/620,153

(22) Filed: Jul. 15, 2003

(30) Foreign Application Priority Data
Jul. 17, 2002 (GB) 0216600.7

(51) Int. Cl. A61M 16/00; B63C 11/02

(52) U.S. Cl. 128/204.26; 128/201.27

(57) ABSTRACT

Self contained underwater breathing apparatus in which a first stage regulator comprises a body (15) defining an internal dry chamber (20), a first port (25) in the chamber for receiving high pressure gas from a supply cylinder, a valve assembly (12, 13, 26) to reduce the high pressure gas to medium pressure gas in the chamber, a second port (29) for delivering the medium pressure gas to a user, and a hydrostatic transmitter (5) responsive to an increase in ambient water pressure and adapted to move within the body (15) accordingly to increase the supply of medium pressure gas to the user, a pressure transducer (9) with a pressure module (4) and a liquid crystal display module (30) visible to the user to indicate the pressure of the medium pressure gas.

THIS INVENTION concerns self-contained underwater breathing apparatus (SCUBA), comprising a gas cylinder providing breathing gas, for example a mixture of nitrogen and oxygen, at a high pressure which is supplied to a first stage gas pressure regulator removably connected to the cylinder outlet and comprising a body defining an internal dry chamber for receiving the high pressure gas and for reducing the pressure thereof through a valve system to provide so-called medium pressure gas which is fed to an outlet port, then via ducting to a second stage gas pressure regulator which acts as a demand valve and is connected to a mouthpiece for delivering gas to the user for breathing.

In such apparatus, the first stage regulator includes means responsive to increased ambient water pressure thus to meet the increased demand for breathing gas, for example, at greater depths.

Typically, the ambient water pressure acts upon a hydrostatic transmitter which interacts with a valve system within the regulator thus to increase the supply of breathing gas to the user.

First stage regulators of this kind have hitherto not included any means whereby the user can check the correct working pressure after it has been set during manufacture or maintenance. Thus the user will wish to check that the correct pressure has been maintained prior to a dive. The means being used for this conventionally is a separate gauge and hose which is connected externally to the regulator to read the pressure, but in use such ancillary equipment is intrusive and connection to the regulator is time-consuming and would normally require the use of a tool.

It is an object of the present invention to provide means contained permanently or non-intrusively within the apparatus whereby the user can easily check for correct breathing gas pressure prior to a dive

According to a first embodiment of the present invention, self contained underwater breathing apparatus, comprises a first stage gas pressure regulator adapted for connection to a breathing gas supply cylinder to reduce the pressure of gas therein to a medium pressure gas and via a hose to a second stage gas pressure regulator to reduce the medium pressure gas to a pressure suitable for breathing; characterised by means contained within the first stage regulator to monitor the pressure of said medium pressure gas and to transmit a signal representative thereof to an indicator housed within the first stage regulator and visible to the user to indicate thereto the pressure of the medium pressure gas.

Preferably the first stage regulator comprises a body defining an internal dry chamber, an inlet port in the chamber of receiving high pressure gas from a source thereof, a valve assembly to reduce the high pressure gas to medium pressure gas in the chamber, an outlet port for delivering the medium pressure gas, and a hydrostatic transmitter responsive to an increase in ambient water pressure and adapted to move within the body accordingly to increase the supply of medium pressure gas to the outlet port, the monitoring means being contained within the body of the first stage regulator.

The monitoring means may be a strain gauge.

The indicator may be means providing a visual read-out, and connected to the strain gauge.

The monitoring means may be associated with the hydrostatic transmitter and movable therewith within the body.

The indicator may be in the form of a pressure module comprising a printed circuit board to which is connected a liquid crystal display.

The pressure module may be connected electrically to the strain gauge by means of a conductor passing along the hydrostatic transmitter whereby the strain gauge and pressure module are mounted on opposed ends thereof respectively.

The pressure module may have a removable transparent cover to protect the electronic circuitry and a battery mounted on and connected to the pressure module.

The removable cover may include a magnifier.

The electronic circuitry may include means whereby the read-out is discontinued after an initial period of indication.

The electronic circuitry and liquid crystal display may provide an indication of the date or a period when a service is due, and/or it may include a real time clock.

According to a second embodiment of the invention, self-contained underwater breathing apparatus comprises a second stage pressure regulator to reduce a medium pressure gas to a pressure suitable for breathing, and adapted for connection to a first stage pressure regulator itself adapted for connection to a breathing gas supply cylinder, the first stage regulator serving to reduce the pressure of gas in the gas supply cylinder to a medium pressure gas; characterised by means contained within the second stage regulator to monitor the pressure of said medium pressure gas and to transmit a signal representative thereof to an indicator housed within the second stage regulator and visible to the user to indicate thereto the pressure of the medium pressure gas.

According to a third embodiment of the invention, self-contained underwater breathing apparatus comprises a hose adapted for connection between a first stage gas pressure regulator to reduce the pressure of gas supplied from a gas supply cylinder to a medium pressure gas, and a second stage gas pressure regulator to reduce the medium pressure gas to a pressure suitable for breathing; characterised by means contained within the hose to monitor the pressure of said medium pressure gas therein and to transmit a signal representative of the medium pressure gas to an indicator housed within the hose and visible to the user to indicate thereto the pressure of the medium pressure gas in the hose.

The first embodiment of the invention will now be described, by way of example only, with reference to the accompanying drawings, in which:—

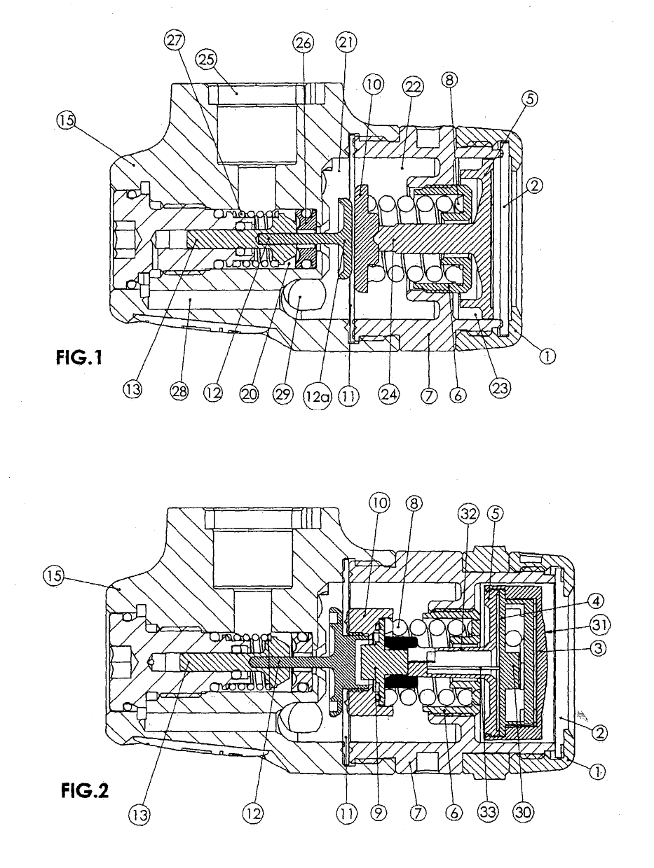

FIG. 1 is a cross-sectional view of a first-stage breathing gas regulator of the conventional kind; and

FIG. 2 is a similar view of a regulator incorporating the invention.

Referring now to FIG. 1, a first stage breathing gas regulator comprises a body 15 defining internal dry chambers 20, 21, 22 and 23. Mounted within the chamber 20 is a high-pressure valve 13 acting upon a valve lifter 12 which passes into chamber 21 and has a head portion 12a which in turn acts upon a diaphragm 11 located between chambers 21 and 22. In chamber 22 and resting against the face of the diaphragm 11 opposite that upon which the valve lifter head 12a rests is a spring-carrier 10 supporting one end of a coil spring 8 the opposite end of which is housed within a spring adjuster 6 threadedly engaged with an internal part of a second body part 7 threadedly attached to the main body part 15.

Slidably engaged within the spring adjuster 6 is the stem 24 of a hydrostatic transmitter 5 maintained within the body part 7 by a threadedly engaged end cap 1. A transparent hydrostatic diaphragm 2 serves to seal the end cap upon the second body part 7.

In use, high pressure supply gas enters the body 15 via an inlet port 25 and is thus contained within the first internal chamber 20 while the high pressure valve 13 rests upon a seat 26. The high pressure valve 13 is retained against the seat 26 by means of a high pressure-valve spring 27.

The second chamber 21 is in communication with a duct 28 leading to an outlet port 29 for delivery of medium pressure gas via a duct, usually a flexible hose, to a second stage regulator and mouthpiece (not shown). The second stage regulator reduces the medium pressure gas to a pressure suitable for breathing on demand.

When the user inhales, the reduced pressure acting on the diaphragm 11 in the chamber 21 causes the spring 8 to overcome the effect of the spring 27, and the valve lifter 12 lifts the high pressure valve 13 off the seat 26 allowing the high pressure gas to pass from the chamber 20 into the chamber 21 and out through duct 28 and port 29.

When the diver exhales, the pressure against diaphragm 11 is restored thus compressing spring 8 and closing the valve.

As the diver descends, the ambient hydrostatic pressure acts upon the hydrostatic diaphragm 2 which in turn acts upon the hydrostatic transmitter 5 thus further opening valve 13 and allowing more breathing gas to pass to the user.

Referring now to FIG. 2, and in accordance with this embodiment of the invention, the hydrostatic transmitter 5 and spring carrier 10 serve to retain a pressure transducer 9 preferably in the form of a strain gauge. Mounted on the outer end of the hydrostatic transmitter 5 is a pressure module 4 comprising electronic circuitry (not shown) and a liquid crystal display module 30. The pressure module 4 also houses a replaceable battery (not shown).

A transparent and protective cover 3 with magnifying curvature, as shown at 31, is mounted over the, pressure module and hydrostatic transmitter 5 to afford protection when the parts are assembled and removed for servicing. As with the regulator illustrated in FIG. 1, the end cap 1 houses a transparent hydrostatic diaphragm 2.

The stem of the hydrostatic transmitter 5, shown at 32, is of tubular construction and contains the electrical connection 33 between the strain gauge 9 and pressure module 4.

The regulator illustrated in FIG. 2 operates in the same manner as the conventional arrangement shown in FIG. 1 save for the provision of the LCD read-out which provides an indication of the pressure of the medium pressure gas available at the regulator.

It is intended that the LCD read-out should be energised upon connection of the regulator to the gas cylinder, and will remain visible for a short period of time so that the user may check the actual pressure of the medium pressure gas. To maximise the battery life the LCD may be arranged to switch off after an initial pre-determined period.

The LCD read-out may comprise a simple indication showing, for example, “high”, “low” and, “OK”. Also included in the electronic circuitry may be means to provide an indication of the date or service period for the equipment. So, for example, after a certain period of time the read-out may show that the equipment requires servicing and/or for example, that the battery condition is low and that the battery therefore needs replacement. If required, other indications may be provided such as the time of day or the period which has elapsed since the equipment was connected to the gas supply. Preferably, minimal information should be provided thus to avoid shortening battery life.

It will be appreciated that by containing user information within the body of a first stage regulator, the need for connecting ancillary measuring equipment is avoided, and the construction of the device is such that it requires only a minimal increase (if any) in size of the regulator when compared with conventional regulators.

Certain physical requirements are imposed upon the pressure module such that, for example, it shall withstand pressure up to 20 bar without sustaining damage while its working pressure may be, for example, 9.5 bar plus or minus 0.5 bar. It must withstand hydrostatic pressure to depths in excess of 50 m and operating temperatures between −40° C. and +60° C. It will be appreciated that the pressure module may be connected to existing regulators with minimal replaceable parts and the device is easily and readily accessible for service procedures.

While the body of a first stage regulator is believed to provide the most convenient location in which the pressure module 4 may be housed, as alternative locations, the body of the second stage regulator, may be used.

Still further, the module may be sufficiently compact and non-intrusive to be connected in-line within the hose connecting the two regulators.

1. Self contained underwater breathing apparatus comprising a first stage gas pressure regulator adapted for connection to a breathing gas supply cylinder to reduce the pressure of gas therein to a medium pressure gas, and via a hose to a second stage gas pressure regulator to reduce the medium pressure gas to a pressure suitable for breathing; characterised by means contained within the first stage regulator to monitor the pressure of said medium pressure gas and to transmit a signal representative thereof to an indicator housed within the first stage regulator and visible to the user to indicate thereto the pressure of the medium pressure gas.

2. Apparatus according to claim 1, wherein the first stage regulator comprises a body defining an internal dry chamber, an inlet port in the chamber for receiving high pressure gas from a source thereof, a valve assembly to reduce the high pressure gas to medium pressure gas in the chamber, an outlet port for the medium pressure gas, and a hydrostatic transmitter responsive to an increase in ambient water pressure and adapted to move within the body accordingly to increase the supply of medium pressure gas to the outlet port, the monitoring means being contained within the body of the first stage regulator.

3. Breathing apparatus according to claim 1, wherein the monitoring means is a strain gauge.

4. Breathing apparatus according to claim 3, wherein the indicator is comprised by means providing a visual read-out, and connected to the strain gauge.

5. Breathing apparatus according to claim 2, wherein the monitoring means is associated with the hydrostatic transmitter and movable therewith within the body.

6. Breathing apparatus according to claim 1, wherein the indicator is in the form of a pressure module comprising a printed circuit board to which is connected a liquid crystal display.

7. Breathing apparatus according to claims 2, wherein the indicator is in the form of a pressure module comprising a printed circuit board to which is connected a liquid crystal display; the monitoring means is a strain gauge; and the pressure module is connected electrically to the strain gauge by means of a conductor passing along the hydrostatic transmitter whereby the strain gauge and pressure module are mounted on opposed ends thereof respectively.

8. Breathing apparatus according to claim 6, wherein the pressure module has a removable transparent cover to protect electronic circuitry of the module, and a battery mounted on and connected to the pressure module.

9. Breathing apparatus according to claim 8, wherein the removable cover includes a magnifier.

10. Breathing apparatus according to claim 6, wherein the pressure module includes electronic circuitry adapted to deactivate the indicator after an initial period of indication.

11. Breathing apparatus according to claim 1, wherein the indicator is adapted to provide an indication of the present date.

12. Breathing apparatus according to claim 1, wherein the indicator is adapted to provide an indication of a period when a service is due.

13. Breathing apparatus according to claim 1, wherein the indicator is adapted to provide an indication of real time.

14. Self-contained underwater breathing apparatus comprising a second stage pressure regulator to reduce a medium pressure gas to a pressure suitable for breathing, and adapted for connection to a first stage pressure regulator itself adapted for connection to a breathing gas supply cylinder, the first stage regulator serving to reduce the pressure of gas in the gas supply cylinder to a medium pressure gas; characterised by means contained within the second stage regulator to monitor the pressure of said medium pressure gas and to transmit a signal representative thereof to an indicator housed within the second stage regulator and visible to the user to indicate thereto the pressure of the medium pressure gas.

15. Self-contained underwater breathing apparatus comprising a hose adapted for connection between a first stage gas pressure regulator to reduce the pressure of gas supplied from a gas supply cylinder to a medium pressure gas, and a second stage gas pressure regulator to reduce the medium pressure gas to a pressure suitable for breathing; characterised by means contained within the hose to monitor the pressure of said medium pressure gas therein and to transmit a signal representative of the medium pressure gas to an indicator housed within the hose and visible to the user to indicate thereto the pressure of the medium pressure gas in the hose.
